(19) United States
(12) Patent Application Publication
(10) Pub. No.: US 2005/0009797 A1
(43) Pub. Date: Jan. 13, 2005

(54) Androstane derivative having substituent in 7- abd 17--positions

(76) Inventors: Mitsuaki Nakamura, Shizuoka (JP); Kazutaka Tachibana, Shizuoka (JP); Ikuhiro Imaoka, Shizuoka (JP); Hitoshi Yoshino, Shizuoka (JP); Setsu Kawata, Shizuoka (JP)

Correspondence Address: BROWDY AND NEIMARK, P.L.L.C.;624 NINTH STREET, NW, SUITE 300, WASHINGTON, DC, 20001-5303, US

(21) Appl. No.: 10/499,044

(22) PCT Filed: Dec. 19, 2002
(86) PCT No.: PCT/JP02/13296

(30) Foreign Application Priority Data
Dec. 19, 2001 (JP) 2001-386302

(51) Int. Cl. A61K 31/56; C07J 1/00

(52) U.S. Cl. 514/178; 552/628

(57) ABSTRACT

A compound of Formula (I): 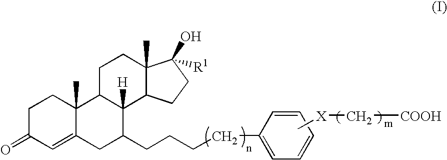
[wherein R¹ represents a lower alkyl group, X represents an oxygen atom or a methylene group, m represents an integer of 1 to 10, and n represents an integer of 0 to 5] or a salt or ester thereof; a medicament and a pharmaceutical composition comprising one or more such compounds; a method for preventing or treating a disease closely related to androgen, which comprises administering an effective amount of one or more such compounds; a kit used for such prevention or treatment; and the use of one or more such compounds for the manufacture of the medicament.

### TECHNICAL FIELD

The present invention relates to 7,17-disubstituted androstane derivatives; medicaments comprising such androstane derivatives; a method for preventing or treating a disease closely related to androgen, which comprises administering an effective amount of one or more such androstane derivatives; a method for preventing or treating a disease selected from the group consisting of prostate cancer, benign prostatic hyperplasia, male pattern alopecia, precocious puberty, acne vulgaris, seborrhea and hypertrichosis, which comprises administering an effective amount of one or more such androstane derivatives; a kit for preventing or treating a disease closely related to androgen, which comprises one or more such androstane derivatives and instructions for use; a kit for preventing or treating a disease selected from the group consisting of prostate cancer, benign prostatic hyperplasia, male pattern alopecia, precocious puberty, acne vulgaris, seborrhea and hypertrichosis, which comprises one or more such androstane derivatives and instructions for use; the use of one or more compounds defined in claim 1 and/or salts and/or esters thereof for the manufacture of an anti-androgen agent; as well as the use of one or more compounds defined in claim 1 and/or salts and/or esters thereof for the manufacture of a prophylactic or therapeutic agent for a disease selected from the group consisting of prostate cancer, benign prostatic hyperplasia, male pattern alopecia, precocious puberty, acne vulgaris, seborrhea and hypertrichosis.

### BACKGROUND ART

The male hormone androgen is known to be closely connected to prostate cancer, benign prostatic hyperplasia, male pattern alopecia, precocious puberty, acne vulgaris, seborrhea and hypertrichosis. For example, it is known that castrated or hypogonadic patients rarely develop prostate cancer or benign prostatic hyperplasia.

As anti-androgen agents (i.e., antagonists against the androgen receptor), various compounds have already been used, including cyproterone acetate, chlormadinone acetate, flutamide and bicalutamide. Cyproterone acetate is known to inhibit the progress of acne and the development of baldness in teenage patients. Cyproterone acetate is also used for the treatment of virilization and alopecia in female patients. Likewise, flutamide and bicalutamide are used as therapeutic agents for prostate cancer.

These anti-androgen agents are effective in many cases including drug therapy for prostate cancer; hence they are regarded as one of the potent therapeutic agents. However, a known problem with these anti-androgen agents is that they will cause recurrence in almost all cases, i.e., most patients will develop androgen resistance two to five years after the agents have proved effective against the disease.

Hydroxyflutamide, an active form of flutamide, is known to enhance the transcriptional activity of androgen receptor at a concentration of 10 μM.

In addition, prostate cancer patients undergoing treatment with flutamide are reported to have blood hydroxyflutamide levels of several μM, which corresponds to a concentration at which hydroxyflutamide shows its agonistic effects (J. Biol. Chem., vol. 270, 19998-20003, 1995). There is also a report indicating that castrated rats continuously administered with cyproterone acetate and chlormadinone acetate for two weeks showed an increase in their prostate weight (Journal of Japan Endocrine Society, vol. 66, 597-606, 1990). Further, flutamide and bicalutamide are reported to have side effects such as liver toxicity.

On the other hand, a 7,11-disubstituted androstane derivative as found in WO01/14406 is a known example of a pure antagonist, i.e., a substance which serves as an antagonist against the androgen receptor without having agonistic effects.

However, the compound of WO01/14406 is less metabolically stable and hence required to be administered at a high dose and/or frequency, thus involving problems of costs, compliance, etc.

### DISCLOSURE OF THE INVENTION

An object of the present invention is to provide a 7,17-disubstituted androstane derivative or a salt or ester thereof.

Another object of the present invention is to provide a medicament comprising the above androstane derivative or a salt or ester thereof.

As a result of research efforts made to achieve the objects stated above, the inventors of the present invention have found that a 7,17-disubstituted androstane derivative or a salt or ester thereof is a pure antagonist against the androgen receptor and is excellent in metabolic stability. This finding led to the completion of the invention.

In a first aspect, the present invention provides a compound of Formula (I): 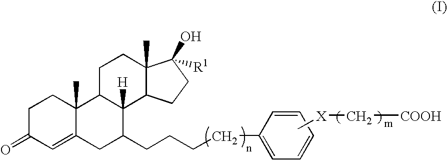
[wherein R¹ represents a lower alkyl group, X represents an oxygen atom or a methylene group, m represents an integer of 1 to 10, and n represents an integer of 0 to 5], or a salt or ester thereof.

In a second aspect, the present invention provides a medicament comprising a compound of Formula (I) and/or a salt and/or ester thereof as an active ingredient.

In a third aspect, the present invention provides a pharmaceutical composition comprising a compound of Formula (I) and/or a salt and/or ester thereof in combination with a pharmaceutically acceptable carrier.

In a fourth aspect, the present invention provides a compound useful as an intermediate for the preparation of a compound of Formula (I) or a salt thereof: 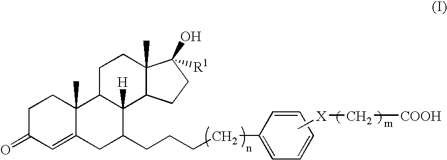
[wherein R¹ represents a lower alkyl group, X represents an oxygen atom or a methylene group, m represents an integer of 1 to 10, and n represents an integer of 0 to 5].

In a fifth aspect, the present invention provides an anti-androgen agent comprising one or more compounds of the above Formula (I) and/or salts and/or esters thereof as an active ingredient.

In a sixth aspect, the present invention provides a prophylactic or therapeutic agent for a disease selected from the group consisting of prostate cancer, benign prostatic hyperplasia, male pattern alopecia, precocious puberty, acne vulgaris, seborrhea and hypertrichosis, which agent comprises one or more compounds of the above Formula (I) and/or salts and/or esters thereof as an active ingredient.

In a seventh aspect, the present invention provides a method for preventing or treating a disease closely related to androgen, which comprises administering to a patient in need thereof, an effective amount of one or more compounds of the above Formula (I) and/or salts and/or esters thereof.

In an eighth aspect, the present invention provides a method for preventing or treating a disease selected from the group consisting of prostate cancer, benign prostatic hyperplasia, male pattern alopecia, precocious puberty, acne vulgaris, seborrhea and hypertrichosis, which method comprises administering to a patient in need thereof, an effective amount of one or more compounds of the above Formula (I) and/or salts and/or esters thereof.

In a ninth aspect, the present invention provides a kit for preventing or treating a disease closely related to androgen, which comprises one or more compounds of the above Formula (I) and/or salts and/or esters thereof, as well as instructions for use.

In a tenth aspect, the present invention provides a kit for preventing or treating a disease selected from the group consisting of prostate cancer, benign prostatic hyperplasia, male pattern alopecia, precocious puberty, acne vulgaris, seborrhea and hypertrichosis, which kit comprises one or more compounds of the above Formula (I) and/or salts and/or esters thereof, as well as instructions for use.

In an eleventh aspect, the present invention provides the use of one or more compounds of the above Formula (I) and/or salts and/or esters thereof for the manufacture of an anti-androgen agent.

In a twelfth aspect, the present invention provides the use of one or more compounds of the above Formula (I) and/or salts and/or esters thereof for the manufacture of a prophylactic or therapeutic agent for a disease selected from the group consisting of prostate cancer, benign prostatic hyperplasia, male pattern alopecia, precocious puberty, acne vulgaris, seborrhea and hypertrichosis.

### BEST MODE FOR CARRYING OUT THE INVENTION

As used herein, a lower alkyl group refers to a linear or branched alkyl group containing 1 to 6 carbon atoms. Examples include a methyl group, an ethyl group, a n-propyl group, an i-propyl group, a n-butyl group, a s-butyl group, an i-butyl group, a t-butyl group, a n-pentyl group, a 3-methylbutyl group, a 2-methylbutyl group, a 1-methylbutyl group, a 1-ethylpropyl group and a n-hexyl group.

In the compound of Formula (I) according to the present invention, the substituent:
—X—(CH₂)ₘ—COOH
is preferably located at the m- or p-position.

In the compound of Formula (I) according to the present invention, the substituent at the 7-position preferably has the α-configuration.

The compound of Formula (I) according to the present invention may be obtained as a salt. Such a salt is preferably a pharmaceutically acceptable salt, examples of which include inorganic base salts such as sodium salt, potassium salt, magnesium salt and zinc salt; and organic base salts such as ammonium salt.

A salt of the compound of Formula (I) according to the present invention may be obtained by reacting the compound of Formula (I) with a base in the presence or absence of a solvent.

Examples of a base include inorganic bases such as sodium hydride, potassium hydride, sodium hydroxide, potassium hydroxide, potassium carbonate and sodium carbonate, as well as organic bases such as ammonia and triethylamine.

Examples of an ester-forming group include linear or branched alkyl groups such as a methyl group, an ethyl group and a n-propyl group; linear or branched alkenyl groups such as an ethenyl group; linear or branched alkynyl groups such as an ethynyl group; cycloalkyl groups such as a cyclohexyl group; aralkyl groups such as a benzyl group; and aryl groups such as a phenyl group. Preferred examples include a methyl group, an ethyl group and a n-propyl group, with a methyl group and an ethyl group being more preferred and an ethyl group being particularly preferred.

A lower alkyl group as R¹ is preferably a methyl group, an ethyl group, a n-propyl group or a n-butyl group, more preferably a methyl group or an ethyl group, and particularly a methyl group.

m is preferably an integer of 1 to 5, more preferably an integer of 1 to 4, and most preferably 3 or 4.

n is preferably an integer of 0 to 3, more preferably an integer of 0 to 2, and most preferably 0 or 1.

The compound of Formula (I) according to the present invention can be prepared, for example, according to Method A or B shown below which may be partially modified as appropriate for the intended compounds.

In the chemical schemes shown in Method A or B, R¹, m, n and X are as defined above, R² represents an ester-forming group, and Y represents a leaving group such as a bromine atom.

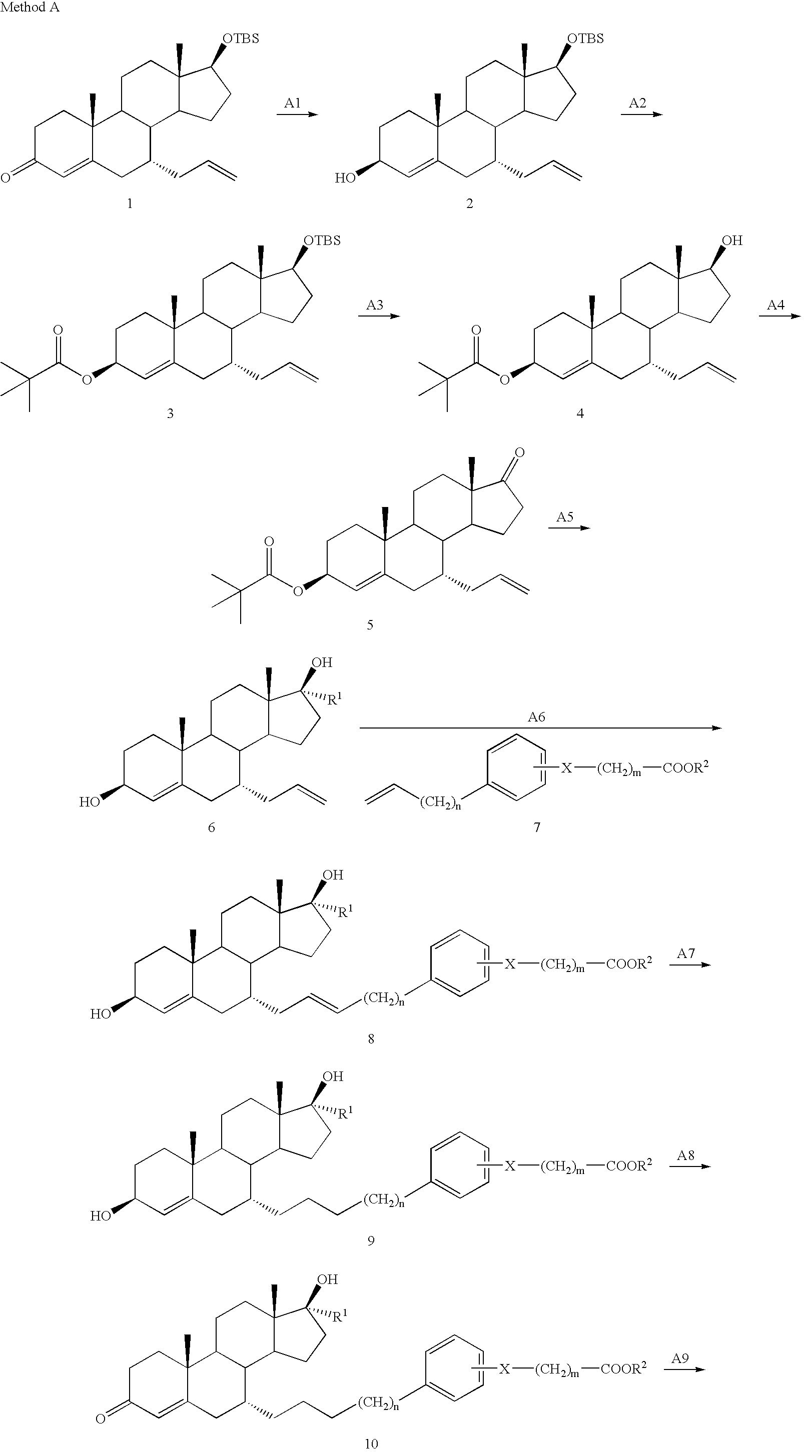

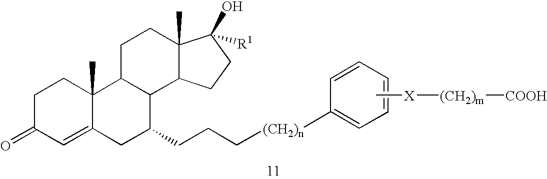

In Step A1, Compound (1) is reacted with a reducing agent in a solvent to prepare Compound (2).

Any solvent may be used as long as it does not inhibit the reaction. Examples include alcohol solvents (e.g., methanol, ethanol, propanol) and ether solvents (e.g., diethyl ether, tetrahydrofuran), with alcohol solvents being preferred and methanol being more preferred.

Examples of a reducing agent available for use include a metal hydride such as sodium borohydride and lithium aluminum hydride, with sodium borohydride being preferred.

The reaction temperature will vary depending on the type of solvent, etc., but usually ranges from −78° C. to 50° C., preferably −10° C. to 30° C.

The reaction time will vary depending on the reaction temperature, etc., but usually ranges from 15 minutes to 24 hours, preferably 30 minutes to 15 hours.

In Step A2, Compound (2) is reacted with pivaloyl chloride in an inert solvent and in the presence of a base to prepare Compound (3).

Any solvent may be used as long as it does not inhibit the reaction. Examples include halogenated solvents such as dichloromethane, with dichloromethane being preferred.

Examples of a base available for use include, but are not limited to, organic bases such as triethylamine, with triethylamine being preferred.

The reaction temperature will vary depending on the type of solvent, etc., but usually ranges from −78° C. to 50° C., preferably −10° C. to 30° C.

The reaction time will vary depending on the reaction temperature, etc., but usually ranges from 15 minutes to 72 hours, preferably 30 minutes to 48 hours.

In Step A3, Compound (3) is reacted with an acid in a solvent such as acetone to prepare Compound (4).

Examples of an acid available for use include, but are not limited to, inorganic acids such as hydrochloric acid and hydrobromic acid, with hydrochloric acid being preferred.

The reaction temperature will vary depending on the type of solvent, etc., but usually ranges from −78° C. to 50° C., preferably −10° C. to 30° C.

The reaction time will vary depending on the reaction temperature, etc., but usually ranges from 15 minutes to 48 hours, preferably 30 minutes to 15 hours.

In Step A4, Compound (4) is reacted with an oxidizing agent in an inert solvent to prepare Compound (5).

Any solvent may be used as long as it does not inhibit the reaction. Examples include halogenated solvents such as dichloromethane, with dichloromethane being preferred.

Examples of an oxidizing agent available for use include, but are not limited to, tetrapropylammonium perruthenate/N-methylmorpholine-N-oxide.

The reaction temperature will vary depending on the type of solvent, etc., but usually ranges from −78° C. to 50° C., preferably −10° C. to 30° C.

The reaction time will vary depending on the reaction temperature, etc., but usually ranges from 15 minutes to 48 hours, preferably 30 minutes to 15 hours.

In Step A5, Compound (5) is reacted with an alkylating agent in an inert solvent to prepare Compound (6).

Any solvent may be used as long as it does not inhibit the reaction. Examples include ether solvents such as tetrahydrofuran, with tetrahydrofuran being preferred.

Examples of an alkylating agent available for use include, but are not limited to, alkyllithiums and alkylmagnesium bromides, with alkyllithiums being preferred. In a case where R¹ is a methyl group, examples of an alkylating agent include, but are not limited to, methyllithium and methylmagnesium bromide, with methyllithium being preferred.

The reaction temperature will vary depending on the type of solvent, etc., but usually ranges from −100° C. to 50° C., preferably −78° C. to 0° C.

The reaction time will vary depending on the reaction temperature, etc., but usually ranges from 15 minutes to 48 hours, preferably 30 minutes to 15 hours.

In Step A6, Compound (6) is reacted with Compound (7) in an inert solvent and in the presence of an organometallic catalyst to prepare Compound (8).

Any inert solvent may be used as long as it does not affect the reaction. Preferred examples include halogenated solvents (e.g., dichloromethane, chloroform), ether solvents (e.g., ether, tetrahydrofuran, dioxane, dimethoxyethane) and aromatic solvents (e.g., benzene, toluene, xylene, quinoline, chlorobenzene), with dichloromethane and dimethoxyethane being more preferred.

An organometallic catalyst available for use is preferably benzylidene-bis(tricyclohexylphosphine)-dichlororuthenium.

The reaction temperature will vary depending on the type of solvent, etc., but usually ranges from −30° C. to 100° C., preferably 0° C. to 80° C.

The reaction time will vary depending on the reaction temperature, etc., but usually ranges from 10 minutes to 72 hours, preferably 30 minutes to 48 hours.

In Step A7, catalytic reduction is performed in an alcohol solvent or in an inert solvent to prepare Compound (9).

Examples of a solvent available for use include alcohol solvents (e.g., methanol, ethanol, n-propanol, i-propanol, n-butanol, s-butanol, t-butanol, pentanol, hexanol, cyclopropanol, cyclobutanol, cyclopentanol, cyclohexanol, ethylene glycol, 1,3-propanediol, 1,4-butanediol, 1,5-pentanediol), ether solvents (e.g., ether, tetrahydrofuran, dioxane, dimethoxyethane), aromatic solvents (e.g., benzene, toluene, xylene, quinoline, chlorobenzene), halogenated solvents (e.g., dichloromethane, chloroform, carbon tetrachloride), as well as cyclohexane, dimethyl sulfoxide, dimethylacetamide, dimethylimidazolidinone, dimethylformamide, N-methylpyrrolidone, ethyl acetate, acetonitrile and nitromethane. Preferred examples include ethanol, dioxane, benzene and ethyl acetate.

The catalytic reduction may be performed under homogeneous conditions such as using:
hydrogen-chlorotris(triphenylphosphine)rhodium(I),
hydrogen-chlorotris(triparatolylphosphine)rhodium(I),
hydrogen-chlorotris(triparamethoxyphenylphosphine)-rhodium(I),
hydrogen-hydridecarbonyltris(triphenylphosphine)-rhodium(I),
hydrogen-rhodium(II) acetate,
hydrogen-ruthenium(II) acetate,
hydrogen-chlorohydridotris(triphenylphosphine)-ruthenium(II),
hydrogen-carboxylatehydridotris(triphenylphosphine)-ruthenium(II),
hydrogen-carbonylhydridotris(triphenylphosphine)-iridium(I),
hydrogen-platinum(II)-tin chloride complex,
hydrogen-pentacyanocobalt(II) complex,
hydrogen-tricyanobipyridinecobalt(II) complex,
hydrogen-bis(dimethylglyoximate)cobalt(II) complex,
hydrogen-methyl benzoate-tricarbonylchromium complex,
hydrogen-bis(tricarbonylcyclopentadienylchromium),
hydrogen-pentacarbonyliron,
hydrogen-bis(cyclopentadienyl)dicarbonyltitanium,
hydrogen-hydridecarbonylcobalt complex,
hydrogen-octacarbonyldicobalt,
hydrogen-hydridecarbonylrhodium,
hydrogen-chromium(III)acetylacetonate-triisobutyl-aluminum,
hydrogen-cobalt(II)acetylacetonate-triisobutylaluminum, and
hydrogen-nickel(II)-2-hexanoate-triethylaluminum,
   or under heterogeneous conditions such as using:
hydrogen-platinum dioxide,
hydrogen-platinum/carbon,
hydrogen-palladium/carbon,
hydrogen-palladium/barium sulfate,
hydrogen-palladium/calcium carbonate,
hydrogen-Raney Nickel,
hydrogen-copper chromite,
hydrogen-rhodium/carbon,
hydrogen-rhodium/alumina,
hydrogen-ruthenium dioxide, and
hydrogen-ruthenium/carbon.

Preferably, the catalytic reduction is performed using hydrogen-chlorotris(triphenylphosphine)rhodium(I), hydrogen-palladium/carbon, hydrogen-palladium/calcium carbonate or the like.

The reaction temperature usually ranges from 0° C. to 100° C., preferably 0° C. to 60° C.

The reaction time will vary depending on the reaction temperature, etc., but usually ranges from 10 minutes to 24 hours, preferably 10 minutes to 6 hours.

In Step A8, Compound (9) is reacted with an oxidizing agent in an inert solvent such as acetone to prepare Compound (10).

Examples of an oxidizing agent available for use include metal oxides such as manganese dioxide. Such an oxidizing agent is preferably a metal oxide, more preferably manganese dioxide.

The reaction temperature usually ranges from 0° C. to 100° C., preferably 0° C. to 60° C.

The reaction time will vary depending on the reaction temperature, etc., but usually ranges from 10 minutes to 48 hours, preferably 10 minutes to 24 hours.

In Step A9, Compound (10) is base-hydrolyzed in a solvent to prepare Compound (11).

Examples of a solvent available for use include, but are not limited to, combinations of water and an alcohol solvent (e.g., methanol) or an ether solvent (e.g., tetrahydrofuran), with a methanol/tetrahydrofuran/water mixture being preferred.

Examples of a base available for use include inorganic bases such as sodium hydroxide, with sodium hydroxide being preferred.

The reaction temperature usually ranges from 0° C. to 100° C., preferably 0° C. to 60° C.

The reaction time will vary depending on the reaction temperature, etc., but usually ranges from 10 minutes to 48 hours, preferably 10 minutes to 24 hours.

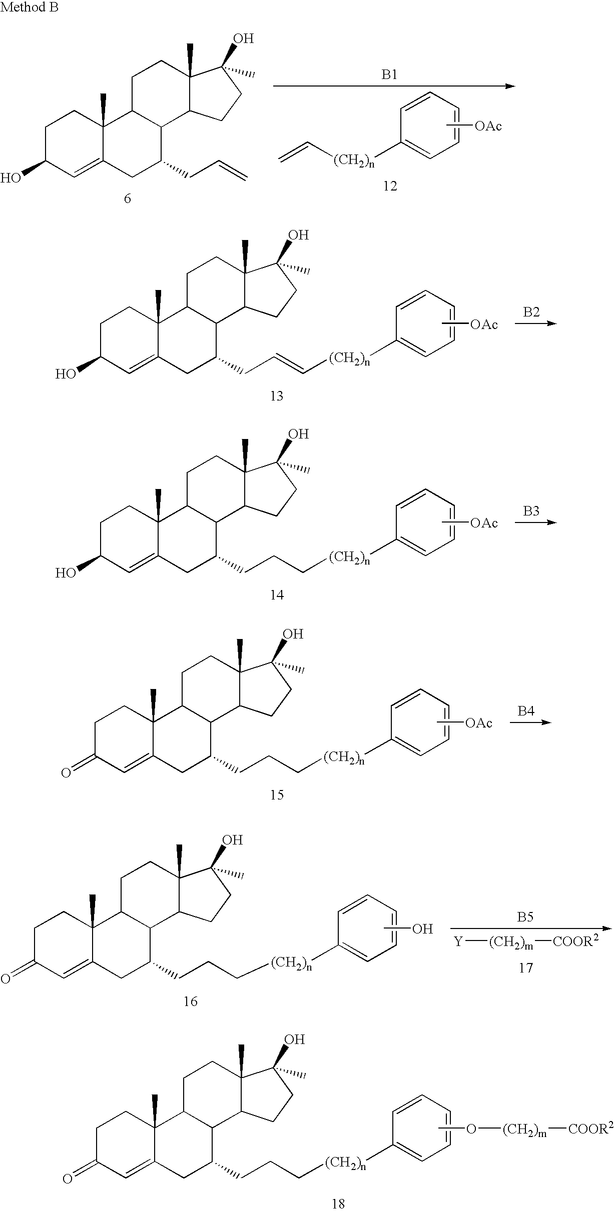

Method B is an alternative method for preparing Compound (18), i.e., Compound (10) wherein X is an oxygen atom.

In Step B1, Compound (6) is reacted with Compound (12) in an inert solvent and in the presence of an organometallic catalyst to prepare Compound (13). This step may be performed in the same manner as Step A6 in Method A.

In Step B2, catalytic reduction is performed in an alcohol solvent or in an inert solvent to prepare Compound (14). This step may be performed in the same manner as Step A7 in Method A.

In Step B3, Compound (6) is reacted with an oxidizing agent (preferably manganese dioxide) in an inert solvent such as acetone to prepare Compound (15). This step may be performed in the same manner as Step A8 in Method A.

In Step B4, Compound (15) is base-hydrolyzed in a solvent to prepare Compound (16). This step may be performed in the same manner as Step A9 in Method A.

In Step B5, Compound (16) is reacted with a base in an inert solvent and in the presence or absence (preferably in the presence) of an additive to give a salt of Compound (16), which in turn is reacted with Compound (17) in an inert solvent to prepare Compound (18).

Examples of the leaving group Y include halogen atoms (e.g., a chlorine atom, a bromine atom, an iodine atom), as well as a methanesulfonyloxy group and a p-toluenesulfonyloxy group. Such a leaving group is preferably a halogen atom (e.g., a chlorine atom, a bromine atom, an iodine atom), more preferably a bromine atom or an iodine atom, and most preferably a bromine atom.

Any inert solvent may be used as long as it does not affect the reaction. Examples include halogenated solvents (e.g., dichloromethane, chloroform, carbon tetrachloride), ether solvents (e.g., ether, tetrahydrofuran, dioxane, dimethoxyethane), aromatic solvents (e.g., benzene, toluene, xylene, quinoline, chlorobenzene), as well as cyclohexane, dimethyl sulfoxide, dimethylacetamide, dimethylimidazolidinone, dimethylformamide and N-methylpyrrolidone. Preferred examples include ether solvents (e.g., ether, tetrahydrofuran, dioxane, dimethoxyethane), as well as dimethyl sulfoxide, dimethylacetamide, dimethylimidazolidinone, dimethylformamide and N-methylpyrrolidone.

Examples of a base available for use include carbonates (e.g., potassium carbonate, sodium carbonate), metal hydrides (e.g., sodium hydride, potassium hydride, calcium hydride), alkyllithiums (e.g., methyllithium, ethyllithium, n-butyllithium, t-butyllithium), metal hydroxides (e.g., lithium hydroxide, sodium hydroxide, potassium hydroxide, potassium hydroxide, calcium hydroxide, barium hydroxide, cesium hydroxide), metal amides (e.g., sodium amide, potassium bistrimethylsilylamide, sodium bistrimethylsilylamide, lithium diisopropylamide), amines (e.g., triethylamine, diisopropylethylamine, 1,8-diazabicyclo[5.4.0]-7-undecene, pyridine, dimethylaminopyridine, pyrazine), as well as sodium tetraborate, sodium iodide, lithium hexamethyldisilazide, sodium hexamethyldisilazide and potassium hexamethyldisilazide. Preferred are carbonates such as potassium carbonate and sodium carbonate.

Examples of an additive available for use include crown ethers such as 18-crown-6-ether and 15-crown-5-ether.

The reaction temperature will vary depending on the type of solvent, etc., but usually ranges from −30° C. to 100° C., preferably 0° C. to 70° C.

The reaction time will vary depending on the reaction temperature, etc., but usually ranges from 10 minutes to 48 hours, preferably 30 minutes to 24 hours.

In Method A, Compound (11) can also be obtained from Compound (9) through steps in the following order: Step A9→Step A8. Alternatively, Compound (11) can be obtained from Compound (8) through steps in the following order: Step A9→Step A7→Step A8.

In Method B, Compound (18) can also be obtained from Compound (14) through steps in the following order: Step B4→Step B5→Step B3 or Step B4→Step B3→Step B5.

Compound (1) used as a starting material is known or may be readily prepared in a known manner with or without modifications [see, e.g., Tetrahedron Letters, 29(13), 1533-1536 (1988)].

Compounds (7) and (12) used as starting materials are known or may be readily prepared in a known manner with or without modifications.

Compound (17) used as a starting material is readily commercially available or known, or may be readily prepared in a known manner with or without modifications.

As used herein, a pure antagonist of the androgen receptor refers to a substance that serves as an antagonist against the androgen receptor and that is substantially free from agonistic effects.

In the substance of the present invention that serves as an antagonist against the androgen receptor and is substantially free from agonistic effects, the phrase “substantially free from agonistic effects” means that the substance provides less than a 5-fold increase in transcriptional activity at a concentration of 0.1 nmol/L to 10 μmol/L, as compared to the value obtained in the absence of the substance, as measured by the androgen receptor reporter gene assay shown below.

Twenty-four hours before transfection, 1.0×10⁵ HeLa cells (purchased from Dainippon Pharmaceutical Co., Ltd.) are cultured in a 12-well microplate containing phenol red-free Dulbecco's Modified Eagle Medium (phenol red-free DMEM) supplemented with charcoal-treated FBS (DCC-FBS; 5%). The HeLa cells are then transfected with 500 ng/well MMTV-Luc vector (a luciferase reporter plasmid with the mouse tumor long terminal repeat containing an androgen-responsive element; a vector prepared by modifying a GM-CAT vector purchased from A.T.C.C. (A.T.C.C. No. 67282) to replace its chloramphenicol acetyltransferase gene by the firefly luciferase gene), 100 ng/well pSG5-hAR (a human androgen receptor expression vector carrying the androgen receptor gene under the control of the SV40 promoter) and 5 ng/well Renilla Luc vector (an internal control vector containing the Renilla luciferase gene integrated thereinto). Transfection is carried out in phenol red-free DMEM medium using 3 μL/well lipofectamine (Gibco BRL). Nine hours after transfection, the medium is replaced by phenol red-free DMEM/3% DCC-FBS supplemented with 10 μmol/L of the compound of Formula (I) or a salt or ester thereof according to the present invention. Forty-eight hours after replacement of the medium, the transcriptional activity is determined by a Dual-Luciferase Reporter Assay System (promega) according to the following equation: (transcriptional activity)=(firefly luciferase activity)/(Renilla luciferase activity). To effect this assay, reference may be made to J. Biol. Chem., vol. 270, p. 19998-20003, 1995.

In contrast, the phrase “acting as an antagonist,” as used herein, means that the transcriptional activity induced by 0.1 nmol/L dihydrotestosterone (DHT) is inhibited to 0% to 50% by the substance of any concentration between 0.1 nmol/L and 10 μmol/L, as measured by the androgen receptor reporter gene assay shown below.

Twenty-four hours before transfection, 1.0×10⁵ HeLa cells are cultured in a 12-well microplate containing phenol red-free DMEM/5% DCC-FBS. The HeLa cells are then transfected with 500 ng/well MMTV-Luc vector, 100 ng/well pSG5-hAR and 5 ng/well Renilla Luc vector. Transfection is carried out in phenol red-free DMEM medium using 3 μL/well lipofectamine. Nine hours after transfection, the medium is replaced by phenol red-free DMEM/3% DCC-FBS supplemented with 0.1 nmol/L DHT and 1.0 μmol/L of the compound of Formula (I) or a salt or ester thereof according to the present invention. Forty-eight hours after replacement of the medium, the transcriptional activity is determined by a Dual-Luciferase Reporter Assay System according to the following equation: (transcriptional activity)=(firefly luciferase activity)/(Renilla luciferase activity). To effect this assay, reference may be made to J. Biol. Chem., vol. 270, p. 19998-20003, 1995.

The compound of Formula (I) or a salt or ester thereof according to the present invention (hereinafter also referred to as a “test substance”) can be assayed for its effects including anti-androgenic activity by using the androgen receptor reporter gene assay, which is used to define the phrase “acting as an antagonist” and/or “free from agonistic effects” as used herein, in combination with the following Assays A to F, as appropriate.

Assay A: In vivo Assay in Rats

Assay A-1: Assay for Antagonistic Effects

Castrated rats, when administered with testosterone or dihydrotestosterone, show an increase in their prostate and seminal vesicle weights. A test substance can be assayed for antagonistic effects by determining whether the test substance inhibits the testosterone- or dihydrotestosterone-induced increase in prostate and seminal vesicle weights. To effect the assay, reference may be made to J. Med. Chem., 41:623-639 (1998) and Clinical Report, 29(4):877-885 (1995), etc.

Assay A-2: Assay for Agonistic Effects

Castrated rats are continuously administered with a test substance. After administration, the test substance can be assayed for agonistic effects by determining whether the rats show an increase in the weights of androgen-responsive organs, i.e., prostates and seminal vesicles. To effect the assay, reference may be made to Journal of Japan Endocrine Society, 66:597-606 (1990), etc.

Assay B: Assay Based on Androgen Receptor Dimerization

Assay B-1: Assay Based on the Ability to Inhibit Dimerization

Dihydrotestosterone causes dimerization of the androgen receptor. A test substance can be assayed for antagonistic effects by determining in a gel-shift assay whether the test substance inhibits androgen receptor dimerization. To effect the assay, reference may be made to J. Biol. Chem., 268:19004-19012 (1993), J. Biol. Chem., 270:19998-20003 (1995), etc.

Assay B-2: Assay Based on the Ability to Stimulate Androgen Receptor Dimerization

A test substance can be assayed for agonistic effects by determining in a gel-shift assay whether the test substance stimulates androgen receptor dimerization. To effect the assay, reference may be made to J. Biol. Chem., 268:19004-19012 (1993), J. Biol. Chem., 270:19998-20003 (1995), etc.

Assay C: Assay Based on Ornithine Decarboxylase (ODC) Activity

A test substance can be assayed for agonistic and antagonistic effects by determining whether the test substance increases or decreases androgen-dependent ODC activity. To effect the assay, reference may be made to Anal. Biochem., 113: 352-355 (198), Journal of Japan Endocrine Society, 66:597-606 (1990), etc.

Assay D: Assay Based on the Binding Ability to Androgen Receptor

A test substance can be assayed for antagonistic effects by determining in a binding assay whether the test substance inhibits the binding between androgen receptor and androgen. To effect the assay, reference may be made to Urology, 48:157-163 (1996), J. Biol. Chem., 270:19998-20003 (1995), Clinical Report, 29(4):877-885 (1995), etc.

Assay E: Assay Based on Increases or Decreases in Androgen Receptor Levels

A test substance can be assayed for agonistic and antagonistic effects on the androgen receptor by determining an increase or decrease in the intracellular androgen receptor levels of androgen receptor-expressing cells when treated with the test substance in the presence and absence of androgen. To effect the assay, reference may be made to Endocrinology, 129: 2000-2010 (1991), etc.

Assay F: Assay Based on Nuclear Transport of Androgen Receptor

A test substance can be assayed for agonistic and/or antagonistic effects by treating androgen receptor-expressing cells with the test substance in the presence or absence of androgen and then examining the intracellular localization of androgen receptors by immunohistological staining to determine the presence or absence of androgen receptor nuclear transport and/or the test substance-induced inhibition of androgen receptor nuclear transport. To effect the assay, reference may be made to J. Biol. Chem., 267:968-974 (1992), etc.

The pharmaceutical composition of the present invention, which comprises a compound of Formula (I) or a salt or ester thereof as an active ingredient, may be administered by oral or parenteral route, desirably by oral route. The composition may be formulated into any dosage form suitable for the intended route of administration.

The pharmaceutical composition of the present invention, which comprises a compound of Formula (I) or a salt or ester thereof as an active ingredient, can be formulated using standard formulation techniques to give solid or liquid formulations such as tablets, capsules, granules, powders, syrups, injections or ointments, depending on the intended use. As used herein, the term “carrier” encompasses pharmaceutical excipients, diluents, solubilizers, coloring agents, flavoring agents and other various additives, which may be selected depending on the type of dosage form. Examples of a carrier used for pharmaceutical purposes include solid or liquid materials such as lactose, magnesium stearate, starch, talc, gelatin, agar, pectin, gum arabic, olive oil, sesame oil, ethylene glycol and other materials commonly used.

The pharmaceutical composition of the present invention, which comprises one or more compounds of Formula (I) and/or salts and/or esters thereof as an active ingredient, may be incorporated into such formulations at varying concentrations depending on the type of dosage form, but it is usually desirable to incorporate the pharmaceutical composition at a concentration of 5% to 100% by weight. The pharmaceutical composition of the present invention, which comprises a compound of Formula (I) or a salt or ester thereof as an active ingredient, may be administered in widely varying amounts depending on the type of homeotherm (including human) to be administered, the severity of symptoms, the doctor's discretion, etc. Based on the active ingredient, the pharmaceutical composition is usually administered in an amount of 1 μg/kg to 500 mg/kg per day, preferably 20 μg/kg to 100 mg/kg per day. Also, it may be given as a single dose or in divided doses on a per-day to per-month basis, which may be altered depending on the severity of symptoms and/or the doctor's discretion.

A kit for preventing or treating a disease closely related to androgen comprises an effective amount of one or more compounds of Formula (I) and/or salts and/or esters thereof, as well as a pharmaceutically acceptable carrier and instructions for use.

A kit for preventing or treating a disease selected from the group consisting of prostate cancer, benign prostatic hyperplasia, male pattern alopecia, precocious puberty, acne vulgaris, seborrhea and hypertrichosis comprises an effective amount of one or more compounds of Formula (I) and/or salts and/or esters thereof, as well as a pharmaceutically acceptable carrier and instructions for use.

### EXAMPLES

### Example 1

### Synthesis of 17β-hydroxy-17α-methyl-7α-(4-(3-(4-carboxy-butoxy)phenyl)butyl)-4-androsten-3-one

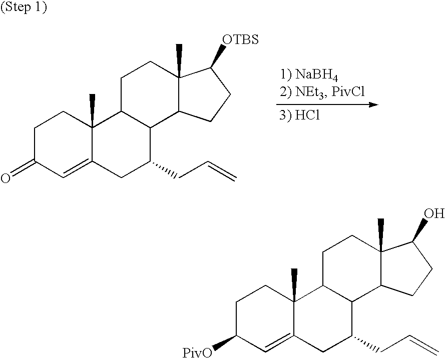

### 17β-Hydroxy-7α-allyl-3β-pivaloyloxy-4-androstene

17β-(t-Butyldimethylsilyloxy)-7α-allyl-4-androsten-3-one (4.1 g) was dissolved in methanol (80 ml). To this solution, sodium borohydride (530 mg) was added in small portions and stirred at room temperature for 30 minutes. After addition of saturated aqueous ammonium chloride, the reaction mixture was extracted with ethyl acetate. The organic layer was washed with saturated brine and then dried over anhydrous magnesium sulfate. After filtration, the solvent was distilled off under reduced pressure to give a crude product (3.9 g). This crude product was dissolved in dichloromethane (50 ml), and triethylamine (50 ml) was added thereto, followed by dropwise addition of pivaloyl chloride (10 ml) with water cooling. The reaction mixture was stirred at room temperature for 2 days. After addition of ethyl acetate, the reaction mixture was washed with 1N hydrochloric acid, saturated aqueous sodium bicarbonate and saturated brine, and then dried over anhydrous magnesium sulfate. After filtration, the solvent was distilled off under reduced pressure to give a crude product (10 g). This crude product was dissolved in acetone (100 ml), and 2N hydrochloric acid was added thereto in small portions, followed by stirring at room temperature for 5 hours. Water was added to the reaction mixture, which was then extracted with ethyl acetate. The organic layer was washed with saturated brine, dried over anhydrous magnesium sulfate and then filtered. After evaporation under reduced pressure to remove the solvent, the resulting residue was purified by silica gel column chromatography (developing solvent: ethyl acetate/n-hexane=1/4) to give the desired product (2.3 g, 61% yield).

¹H-NMR (270 MHz, CDCl₃) δ: 0.77(3H, s), 0.80-2.30(21H, m), 1.10(3H, s), 1.19(9H, s), 3.60-3.70(1H, m), 4.96-5.01(2H, m), 5.14(1H, s), 5.18-5.28(1H, m), 5.55-5.78(1H, m). Rf value: 0.19 (on a silica gel plate, developed with ethyl acetate/hexane=1/4).

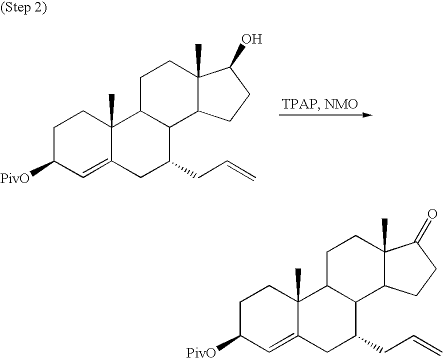

### 7α-Allyl-3β-pivaloyloxy-4-androsten-17-one

17β-Hydroxy-7α-allyl-3β-pivaloyloxy-4-androstene (2.3 g) was dissolved in dichloromethane (180 ml). To this solution, molecular sieve 4A and N-methylmorpholine-N-oxide (2.0 g) were added and stirred at room temperature for 5 minutes. After addition of tetrapropylammonium perruthenate (310 mg), stirring was continued at room temperature for an additional 2 hours. The reaction mixture was filtered through celite, and the filtrate was washed with saturated aqueous sodium sulfite and saturated aqueous ammonium chloride, dried over anhydrous magnesium sulfate and then filtered. After evaporation under reduced pressure to remove the solvent, the resulting residue was purified by silica gel column chromatography (developing solvent: ethyl acetate/n-hexane=1/4) to give the desired product (1.4 g, 59% yield).

¹H-NMR (270 MHz, CDCl₃) δ: 0.90 (3H, s), 1.03-2.33(19H, m), 1.12(3H, s), 1.19(9H, s), 2.47(1H, dd, J=8.2, 21 Hz), 4.97-5.04(2H, m), 5.18(1H, s), 5.20-5.30(1H, m), 5.63-5.80(1H, m). Rf value: 0.52 (on a silica gel plate, developed with ethyl acetate/hexane=1/4).

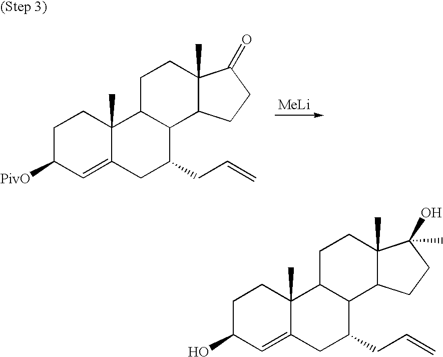

### 17β-Hydroxy-17α-methyl-7α-allyl-3β-hydroxy-4-androstene

Under a nitrogen atmosphere, 7α-allyl-3β-pivaloyloxy-4-androsten-17-one (1.4 g) was dissolved in tetrahydrofuran (55 ml). To this solution, methyllithium in ether (1.14 M, 85 ml) was added dropwise at −78° C. and stirred for 1.5 hours. After addition of saturated aqueous ammonium chloride, the reaction mixture was warmed to room temperature and extracted with ethyl acetate. The organic layer was washed with saturated brine, dried over anhydrous magnesium sulfate and then filtered. After evaporation under reduced pressure to remove the solvent, an ethyl acetate/hexane mixture was added to the resulting crude product, followed by filtration to collect insoluble matter, thereby obtaining the desired product (0.78 g, 70% yield).

¹H-NMR (270 MHz, CDCl₃) δ: 0.88(3H, s), 0.90-2.03(20H, m), 1.10(3H, s), 1.21(3H, s), 2.06(1H, dd, J=1.9, 12 Hz), 2.18-2.28(1H, m), 4.13-4.25(1H, m), 4.95-5.01(2H, m), 5.25(1H, s), 5.60-5.78 (1H, m). Rf value: 0.054 (on a silica gel plate, developed with ethyl acetate/hexane=1/4).

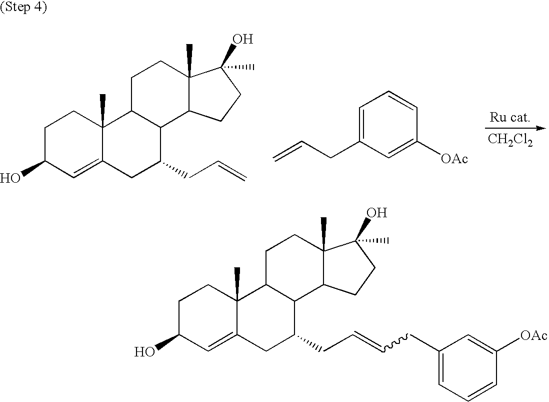

### 17β-Hydroxy-17α-methyl-7α-(4-(3-acetoxyphenyl)-2-buten-1-yl)-3β-hydroxy-4-androstene

17β-Hydroxy-17α-methyl-7α-allyl-3β-hydroxy-4-androstene (147 mg) and 1-acetoxy-3-allylbenzene (158 mg) were dissolved in dichloromethane (4 ml) and then heated at reflux for 7 hours under an argon atmosphere in the presence of benzylidene-bis(tricyclohexylphosphine)dichlororuthenium (20 mg). After cooling, the reaction mixture was concentrated under reduced pressure and purified by silica gel column chromatography (developing solvent: ethyl acetate/n-hexane=1/2) to give the desired product (135 mg, 64% yield).

¹H-NMR (270 MHz, CDCl₃) δ: 0.88(3H, s), 0.90-2.25(22H, m), 1.08(3H, s), 1.19(3H, s), 2.30(3H, s), 3.34(2H, d, J=6.6 Hz), 4.10-4.20(1H, m), 5.12(1H, s), 5.30-5.58(2H, m), 6.89-6.91(2H, m), 7.05-7.09(1H, m), 7.25-7.31(1H, m). Rf value: 0.13 (on a silica gel plate, developed with ethyl acetate/hexane=1/2).

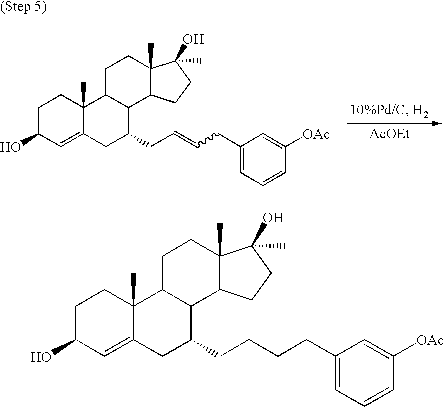

### 17β-Hydroxy-17α-methyl-7α-(4-(3-acetoxyphenyl)butyl)-3β-hydroxy-4-androstene

17β-Hydroxy-17α-methyl-7α-(4-(3-acetoxyphenyl)-2-buten-1-yl)-3β-hydroxy-4-androstene (135 mg) was dissolved in ethyl acetate and then stirred at room temperature for 4 hours under a hydrogen atmosphere in the presence of 10%-Pd/C. After the reaction mixture was filtered, the filtrate was concentrated under reduced pressure and then purified by silica gel column chromatography (developing solvent: ethyl acetate/n-hexane=1/2 to 1/1) to give the desired product (93 mg, 69% yield).

¹H-NMR (270 MHz, CDCl₃) δ: 0.87(3H, s), 0.90-2.05(26H, m), 1.08(3H, s), 1.21(3H, s), 2.18-2.20(1H, m), 2.29(3H, s), 2.61(2H, t, J=7.1 Hz), 4.10-4.22(1H, m), 5.17(1H, s), 6.87-6.93(2H, m), 7.04(1H, d, J=8.1 Hz), 7.24-7.31(1H, m). Rf value: 0.51 (on a silica gel plate, developed four times with ethyl acetate/hexane=1/2).

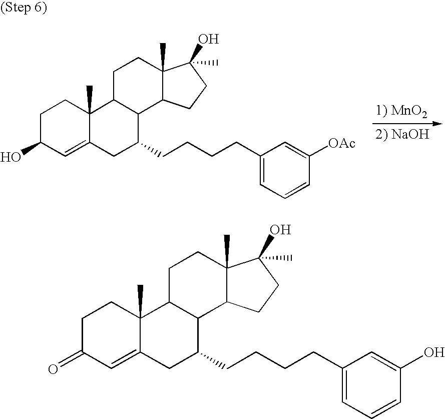

### 17β-Hydroxy-17α-methyl-7α-(4-(3-hydroxyphenyl)butyl)-4-androsten-3-one

17β-Hydroxy-17α-methyl-7α-(4-(3-acetoxyphenyl)butyl)-3β-hydroxy-4-androstene (93 mg) was dissolved in acetone (10 ml) and then stirred overnight at room temperature in the presence of manganese dioxide (305 mg). After further addition of manganese dioxide (300 mg), stirring was continued overnight at room temperature. The reaction mixture was filtered through celite, and the filtrate was concentrated under reduced pressure. The resulting residue was dissolved in methanol (2 ml), followed by addition of 2N NaOH (0.3 ml) and water (1 ml), and then stirred at room temperature for 2 hours. After addition of saturated aqueous ammonium chloride, the reaction mixture was extracted with ethyl acetate. The organic layer was washed with saturated brine, dried over anhydrous magnesium sulfate and then filtered. After evaporation under reduced pressure to remove the solvent, the resulting residue was purified by silica gel column chromatography (developing solvent: ethyl acetate/n-hexane=1/1) to give the desired product (84 mg, 100% yield).

¹H-NMR (270 MHz, CDCl₃) δ: 0.91(3H, s), 0.95-1.90(20H, m), 1.24(3H, s), 1.25(3H, s), 2.07-2.73(7H, m), 5.75(1H, s), 6.45(1H, s), 6.68-6.73(2H, m), 7.17(1H, t, J=7.8 Hz), 7.57(1H, s). Rf value: 0.50 (on a silica gel plate, developed twice with ethyl acetate/hexane=1/1). Mass (ESI): 451.4[M+H].

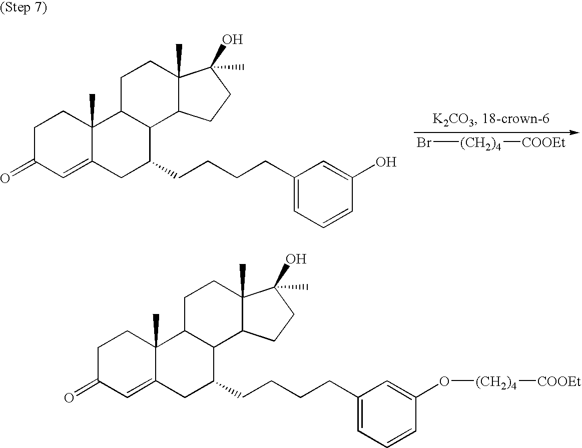

### 17β-Hydroxy-17α-methyl-7α-(4-(3-(4-ethoxycarbonylbutoxy)-phenyl)butyl)-4-androsten-3-one

17β-Hydroxy-17α-methyl-7α-(4-(3-hydroxyphenyl)butyl)-4-androsten-3-one (23 mg), ethyl 5-bromovalerate (0.0165 ml) and 18-crown-6-ether (13 mg) were dissolved in N,N-dimethyl-acetamide (0.5 ml). To this solution, potassium carbonate (16 mg) was added and stirred at room temperature for 3 hours under an argon atmosphere. After addition of saturated aqueous ammonium chloride, the reaction mixture was extracted with ethyl acetate. The organic layer was washed with saturated brine, dried over anhydrous magnesium sulfate and then filtered. After evaporation under reduced pressure to remove the solvent, the resulting residue was purified on a silica gel plate (developing solvent: ethyl acetate/n-hexane=1/1) to give the desired product (23 mg, 76% yield).

¹H-NMR (270 MHz, CDCl₃) δ: 0.90(3H, s), 1.00-2.65(33H, m), 1.21(3H, s), 1.23(3H, s), 1.26(3H, t, J=7.1 Hz), 3.90-4.00(2H, m), 4.13(2H, q, J=7.1 Hz), 5.71(1H, s), 6.69-6.75(3H, m), 7.15-7.20(1H, m). Rf value: 0.32 (on a silica gel plate, developed with ethyl acetate/hexane=1/1)

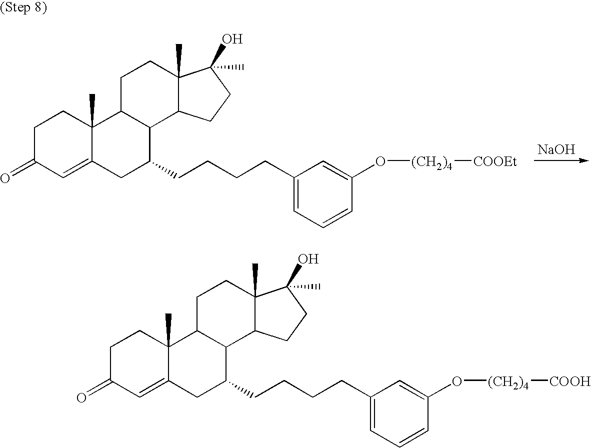

### 17β-Hydroxy-17α-methyl-7α-(4-(3-(4-carboxybutoxy)phenyl)-butyl)-4-androsten-3-one

17β-Hydroxy-17α-methyl-7α-(4-(3-(4-ethoxycarbonyl-butoxy)phenyl)butyl)-4-androsten-3-one (23 mg) was dissolved in methanol (1 ml). To this solution, 2N NaOH (0.5 ml), water (1 ml) and tetrahydrofuran (0.5 ml) were added and stirred at room temperature for 3 hours. After addition of saturated aqueous ammonium chloride, the reaction mixture was extracted with ethyl acetate. The organic layer was washed with saturated brine, dried over anhydrous magnesium sulfate and then filtered. After evaporation under reduced pressure to remove the solvent, the resulting residue was purified on a silica gel plate (developing solvent: methanol/chloroform=1/10) to give the desired product (19 mg, 86% yield).

¹H-NMR (270 MHz, CDCl₃) δ: 0.90(3H, s), 1.00-2.65(33H, m), 1.21(3H, s), 1.23(3H, s), 3.90-4.00(2H, m), 5.72(1H, s), 6.67-6.78(3H, m), 7.14-7.20(1H, m). Rf value: 0.24 (on a silica gel plate, developed with methanol/chloroform=1/10). Mass(ESI): 551.4[M+H].

The same procedure as shown in Example 1 was repeated to synthesize the following compounds.

TABLE 1

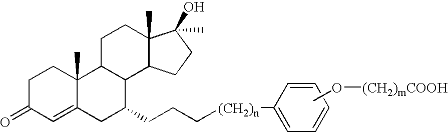

| | | Substituent | | |
|---|---|---|---|---|
| Example | n | position | m | Physical property data |
| 2 | 0 | p | 4 | Mass(ESI): 537.3[M + H] |
| | | | | Rf value: 0.10 (on a silica |
| | | | | gel plate, developed with |
| | | | | ethyl acetate/hexane = 1/1) |
| 3 | 0 | p | 5 | Mass(ESI): 551.3[M + H] |
| 4 | 1 | p | 3 | Mass(ESI): 519.4[M + H − 18] |
| | | | | Rf value: 0.038(on a silica |
| | | | | gel plate, developed with |
| | | | | ethyl acetate/hexane = 1/1) |
| 5 | 1 | p | 4 | Mass(ESI): 533.4[M + H − 18] |
| | | | | Rf value: 0.067 (on a silica |
| | | | | gel plate, developed with |
| | | | | ethyl acetate/hexane = 1/1) |
| 6 | 1 | p | 5 | Mass(ESI): 547.3[M + H − 18] |
| | | | | Rf value: 0.050(on a silica |
| | | | | gel plate, developed with |
| | | | | ethyl acetate/hexane = 1/1) |
| 7 | 2 | p | 1 | Mass(ESI): 523.4[M + H] |
| | | | | Rf value: 0.10(on a silica |
| | | | | gel plate, developed with |
| | | | | methanol/chloroform = 1/10) |
| 8 | 2 | p | 3 | Mass(ESI): 533.3[M + H − 18] |
| | | | | Rf value: 0.38(on a silica |
| | | | | gel plate, developed with |
| | | | | methanol/chloroform = 1/10) |
| 9 | 2 | p | 4 | Mass(ESI): 547.4[M + H − 18] |
| | | | | Rf value: 0.38(on a silica |
| | | | | gel plate, developed with |
| | | | | methanol/chloroform = 1/10) |
| 10 | 2 | p | 5 | Mass(ESI): 579.3[M + H] |
| | | | | Rf value: 0.43(on a silica |
| | | | | gel plate, developed with |
| | | | | methanol/chloroform = 1/10) |
| 11 | 1 | m | 1 | Mass(ESI): 509.5[M + H] |
| | | | | Rf value: 0.042(on a silica |
| | | | | gel plate, developed with |
| | | | | methanol/chloroform = 1/10) |
| 12 | 1 | m | 3 | Mass(ESI): 519.4[M + H − 18] |
| | | | | Rf value: 0.30(on a silica |
| | | | | gel plate, developed with |
| | | | | methanol/chloroform = 1/10) |
| 13 | 1 | m | 5 | Mass(ESI): 565.4[M + H] |
| | | | | Rf value: 0.24(on a silica |
| | | | | gel plate, developed with |
| | | | | methanol/chloroform = 1/10) |
| 14 | 2 | m | 1 | Mass(ESI): 523.4[M + H] |
| 15 | 2 | m | 3 | Mass(ESI): 551.4[M + H] |
| 16 | 2 | m | 4 | Mass(ESI): 565.4[M + H] |
| 17 | 2 | m | 5 | Mass(ESI): 561.4[M + H − 18] |

### Example 18

### Synthesis of 17β-hydroxy-17α-methyl-7α-(3-(4-(3-carboxy-propoxy)phenyl)propyl)-4-androsten-3-one

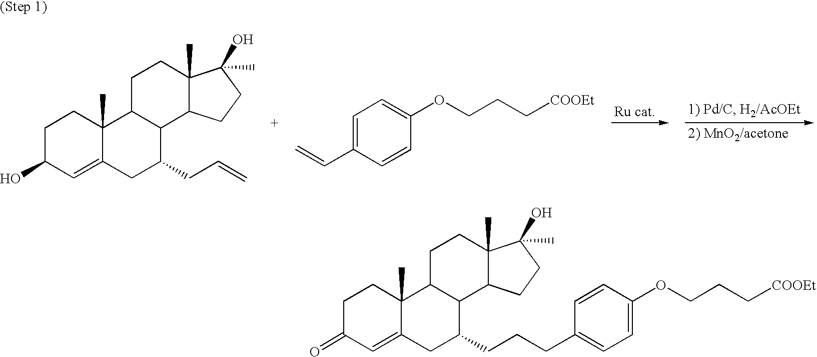

### 17β-Hydroxy-17α-methyl-7α-(3-(4-(3-ethoxycarbonylpropoxy)-phenyl)propyl)-4-androsten-3-one

17β-Hydroxy-17α-methyl-7α-allyl-3β-hydroxy-4-androstene (302 mg) and 4-(3-ethoxycarbonylpropoxy)styrene (414 mg) were dissolved in dichloromethane (7 ml) and then heated overnight at reflux under an argon atmosphere in the presence of benzylidene-bis(tricyclohexylphosphine)dichlororuthenium (49 mg). After cooling, the reaction mixture was concentrated under reduced pressure and then purified by silica gel column chromatography (developing solvent: ethyl acetate/n-hexane=1/2). The resulting product was dissolved in ethyl acetate (20 ml) and then stirred at room temperature for 1.5 hours under a hydrogen atmosphere in the presence of 10%-Pd/C (95 mg). After the reaction mixture was filtered and concentrated under reduced pressure, the resulting residue was dissolved in acetone (20 ml) and stirred overnight at room temperature in the presence of manganese dioxide (558 mg). The reaction mixture was filtered through celite, and the filtrate was concentrated under reduced pressure and then purified by silica gel column chromatography (developing solvent: ethyl acetate/n-hexane=1/2 to 1/1) to give the desired product (255 mg, 53% yield).

¹H-NMR (270 MHz, CDCl₃) δ: 0.90(3H, s), 1.00-2.57(29H, m), 1.21(6H, s), 1.26(3H, t, J=7.1 Hz), 3.98(2H, t, J=5.9 Hz), 4.14(2H, q, J=7.1 Hz), 5.70(1H, s), 6.80(2H, d, J=8.6 Hz), 7.05(2H, d, J=8.6 Hz). Rf value: 0.25 (on a silica gel plate, developed twice with ethyl acetate/hexane=1/2).

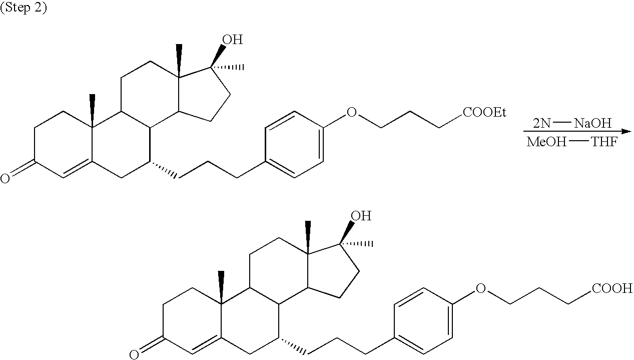

### 17β-Hydroxy-17α-methyl-7α-(3-(4-(3-carboxypropoxy)phenyl)-propyl)-4-androsten-3-one

17β-Hydroxy-17α-methyl-7α-(3-(3-(3-ethoxycarbonyl-propoxy)phenyl)propyl)-4-androsten-3-one (251 mg) was dissolved in methanol (5 ml) and tetrahydrofuran (2 ml). To this solution, 2N NaOH (2 ml) was added and stirred at room temperature for 2.5 hours. After addition of saturated aqueous ammonium chloride, the reaction mixture was extracted with ethyl acetate. The organic layer was washed with saturated brine, dried over anhydrous magnesium sulfate and then filtered. After evaporation under reduced pressure to remove the solvent, the resulting residue was purified on a silica gel plate (developing solvent: methanol/chloroform=1/30 to 1/20) to give the desired product (242 mg, 100% yield).

¹H-NMR (270 MHz, CDCl₃) δ: 0.90(3H, s), 1.00-2.63(29H, m), 1.20(6H, s), 4.00(2H, t, J=5.8 Hz), 5.66(1H, s), 6.80(2H, d, J=8.6 Hz), 7.05(2H, d, J=8.6 Hz). Rf value: 0.25 (on a silica gel plate, developed with methanol/chloroform=1/10). Mass(ESI): 523.3[M+H].

The same procedure as shown in Example 18 was repeated to synthesize the following compounds.

TABLE 2

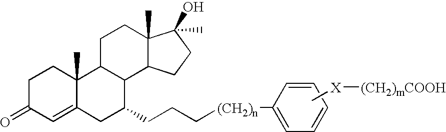

| | | Substituent | | | |
|---|---|---|---|---|---|
| Example | n | position | X | m | Physical property data |
| 19 | 0 | p | O | 2 | Mass(ESI): 509.3[M + H] |
| | | | | | Rf value: 0.017(on a |
| | | | | | silica gel plate, |
| | | | | | developed with ethyl |
| | | | | | acetate/hexane = 1/1) |
| 20 | 0 | m | O | 3 | Mass(ESI): 523.3[M + H] |
| | | | | | Rf value: 0.067(on a |
| | | | | | silica gel plate, |
| | | | | | developed with ethyl |
| | | | | | acetate/hexane = 1/1) |
| 21 | 0 | p | CH₂ | 2 | Mass(ESI): 507.3[M + H] |
| | | | | | Rf value: 0.083(on a |
| | | | | | silica gel plate, |
| | | | | | developed with ethyl |
| | | | | | acetate/hexane = 1/1) |
| 22 | 0 | p | CH₂ | 3 | Mass(ESI): 521.4[M + H] |
| | | | | | Rf value: 0.38(on a |
| | | | | | silica gel plate, |
| | | | | | developed with |
| | | | | | methanol/chloroform = |
| | | | | | 1/10) |
| 23 | 0 | p | CH₂ | 4 | Mass(ESI): 535.3[M + H] |
| | | | | | Rf value: 0.067(on a |
| | | | | | silica gel plate, |
| | | | | | developed with ethyl |
| | | | | | acetate/hexane = 1/1) |
| 24 | 0 | m | CH₂ | 3 | Mass(ESI): 521.3[M + H] |
| | | | | | Rf value: 0.31(on a |
| | | | | | silica gel plate, |
| | | | | | developed with |
| | | | | | methanol/chloroform = |
| | | | | | 1/10) |

### Example 25

### Examination of Test Compounds for Agonistic Effects

Twenty-four hours before transfection, 1.0×10⁵ HeLa cells were cultured in a 12-well microplate containing phenol red-free DMEM/5% DCC-FBS. The HeLa cells were then transfected with 500 ng/well MMTV-Luc vector, 100 ng/well pSG5-hAR and 5 ng/well Renilla Luc vector. Transfection was carried out in phenol red-free DMEM medium using 3 mL/well lipofectamine. Nine hours after transfection, the medium was replaced by phenol red-free DMEM/3% DCC-FBS supplemented with a test compound (1, 10, 100, 1000 or 10000 nmol/L). Forty-eight hours after replacement of the medium, the transcriptional activity was determined by a Dual-Luciferase Reporter Assay System according to the following equation:
(transcriptional activity)=(firefly luciferase activity)/(Renilla luciferase activity).

The transcriptional activity thus determined was used to calculate agonist activity according to the following equation, which in turn was used to calculate a FI5 value (i.e., a concentration at which the compound provides a 5-fold increase in transcriptional activity as compared to the value obtained in the absence of the compound).
Agonist activity=(transcriptional activity in the presence of the compound)/(transcriptional activity in the absence of the compound)

### Example 26

### Examination of Test Compounds for Antagonistic Effects

Twenty-four hours before transfection, 1.0×10⁵ HeLa cells were cultured in a 12-well microplate containing phenol red-free DMEM/5% DCC-FBS. The HeLa cells were then transfected with 500 ng/well MMTV-Luc vector, 100 ng/well pSG5-hAR and 5 ng/well Renilla Luc vector. Transfection was carried out in phenol red-free DMEM medium using 3 mL/well lipofectamine. Nine hours after transfection, the medium was replaced by phenol red-free DMEM/3% DCC-FBS supplemented with DHT (0.1 nmol/L) and a test compound (1, 10, 100, 1000 or 10000 nmol/L). Forty-eight hours after replacement of the medium, the transcriptional activity was determined by a Dual-Luciferase Reporter Assay System according to the following equation: (transcriptional activity)=(firefly luciferase activity)/(Renilla luciferase activity).

The transcriptional activity thus determined was used to calculate antagonist activity according to the following equation, which in turn was used to calculate a IC50 value (i.e., a concentration at which the compound causes 50% inhibition of transcriptional activity induced by DHT (0.1 nmol/L) in the absence of the compound).
Antagonist activity=[(transcriptional activity in the presence of the compound)/(transcriptional activity in the absence of the compound)]×100

Table 3 shows the results obtained in Examples 25 and 26.

TABLE 3

| Test compound | IC50 value (nM) | FI5 value (nM) |
|---|---|---|
| Compound from Example 1 | 333 | ND* |
| Compound from Example 7 | 5136 | ND |
| Compound from Example 12 | 921 | ND |
| Compound from Example 13 | 529 | ND |
| Compound from Example 18 | 664 | ND |
| Compound from Example 20 | 815 | ND |
| Compound from Example 22 | 717 | ND |
| Compound from Example 24 | 4037 | ND |

ND* in the table means that a FI5 value cannot be calculated because the compound provides less than a 5-fold increase in transcriptional activity even at a concentration of 10000 nM.

In view of the above test results, the compound of the present invention was confirmed to be substantially free from agonistic effects on androgen receptor-mediated transcriptional activation. This suggested that the compound of the present invention could reduce the risk of developing androgen resistance, which was observed in anti-androgen agents conventionally used.

### Example 27

### Examination of Metabolic Stability

A test tube containing 0.1 mol/L potassium phosphate buffer (345 μL, pH 7.4) was cooled on ice, followed by addition of 5 mg/mL rat liver microsome (50 μL), 0.1 mol/L magnesium chloride (50 μL) and a test compound dissolved in ethanol (adjusted to 1 mg/mL, 5 μL). After pre-incubation at 37° C. for 5 minutes, 10 mM β-Nicotinamide-adenine dinucleotide, oxidized form (β-NAD⁺) was added to initiate the reaction. The reaction was continued at 37° C. for 0, 5 and 15 minutes, followed by addition of 0.1 mol/L hydrochloric acid (100 μL) and diethyl ether (5 mL) to stop the reaction. The reaction mixture was stirred for 10 minutes and then centrifuged at 3000 rpm for 10 minutes. The upper organic layer (4.5 mL) was transferred to another test tube, evaporated to dryness under a nitrogen stream, dissolved in 40% acetonitrile (200 μL) and then measured for the peak area of the test compound by high performance liquid chromatography. The residual percentage of the test compound at a given point in time was calculated using the following equation:
Residual (%)=[(peak area of the test compound at a given time point)/(peak area of the test compound at 0 minutes)]×100

Table 4 shows the results obtained in Example 27.

TABLE 4

Residual (%)

| Test compound | 5 minutes | 15 minutes |
|---|---|---|
| Example 18 | 113.7 | 110.8 |
| Comparative Example* | 59.4 | 15.1 |

Comparative Example*:
17β-hydroxy-7α-{7-(N,N-dimethylaminocarbonyl)heptyl}-5α-androstan-3-one (which is disclosed in WO01/14406)

In view of the above test results, the compound of the present invention was confirmed to be resistant to metabolism in the presence of β-NAD⁺. This suggested that the compound of the present invention was more stable in vivo and hence more pharmacologically active than the compound of WO01/14406.

Industrial Applicability

The compound of Formula (I) or a salt or ester thereof according to the present invention is expected to provide an anti-androgen agent without the risk of developing androgen resistance due to prolonged administration and/or without the risk of side effects including liver toxicity. The compound of the present invention is therefore expected to be effective in preparing pharmaceutical compositions, e.g., therapeutic agents for diseases such as prostate cancer, benign prostatic hyperplasia, male pattern alopecia, precocious puberty, acne vulgaris, seborrhea and hypertrichosis. When pre-administered, the compound of Formula (I) or a salt or ester thereof according to the present invention is also expected to prevent or delay the onset of diseases such as prostate cancer, benign prostatic hyperplasia, male pattern alopecia, precocious puberty, acne vulgaris, seborrhea and hypertrichosis; it is therefore expected to provide prophylactic agents for these diseases. Moreover, since the compound of Formula (I) or a salt or ester thereof according to the present invention is metabolically stable, it is possible to reduce the dose required and also to save preparation costs. In addition, since the compound of the present invention can also be administered at a reduced frequency, it is convenient for patients and is expected to provide improvements in compliance.

1. A compound of Formula (I): 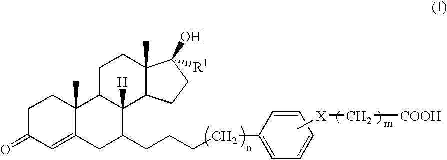
[wherein R¹ represents a lower alkyl group, X represents an oxygen atom or a methylene group, m represents an integer of 1 to 10, and n represents an integer of 0 to 5], or a salt or ester thereof:

2. The compound according to claim 1, wherein R¹ is a methyl group, or a salt or ester thereof.

3. A medicament comprising one or more compounds according to claim 1 and/or salts and/or esters thereof as an active ingredient.

4. A pharmaceutical composition comprising one or more compounds according to claim 1 and/or salts and/or esters thereof in combination with a pharmaceutically acceptable carrier.

5. An anti-androgen agent comprising one or more compounds according to claim 1 and/or salts and/or esters thereof as an active ingredient.

6. A prophylactic or therapeutic agent for a disease selected from the group consisting of prostate cancer, benign prostatic hyperplasia, male pattern alopecia, precocious puberty, acne vulgaris, seborrhea and hypertrichosis, which comprises one or more compounds according to claim 1 and/or salts and/or esters thereof as an active ingredient.

7. A method for preventing or treating a disease closely related to androgen, which comprises administering to a patient in need thereof, an effective amount of one or more compounds according to claim 1 and/or salts and/or esters thereof.

8. A method for preventing or treating a disease selected from the group consisting of prostate cancer, benign prostatic hyperplasia, male pattern alopecia, precocious puberty, acne vulgaris, seborrhea and hypertrichosis, which comprises administering to a patient in need thereof, an effective amount of one or more compounds according to claim 1 and/or salts and/or esters thereof.

9. A kit for preventing or treating a disease closely related to androgen, which comprises an effective amount of one or more compounds according to claim 1 and/or salts and/or esters thereof, as well as instructions for use.

10. A kit for preventing or treating a disease selected from the group consisting of prostate cancer, benign prostatic hyperplasia, male pattern alopecia, precocious puberty, acne vulgaris, seborrhea and hypertrichosis, which comprises an effective amount of one or more compounds according to claim 1 and/or salts and/or esters thereof, as well as instructions for use.

11. The use of one or more compounds according to claim 1 and/or salts and/or esters thereof for the manufacture of an anti-androgen agent.

12. The use of one or more compounds according to claim 1 and/or salts and/or esters thereof for the manufacture of a prophylactic or therapeutic agent for a disease selected from the group consisting of prostate cancer, benign prostatic hyperplasia, male pattern alopecia, precocious puberty, acne vulgaris, seborrhea and hypertrichosis.
